(19) United States
(12) Patent Application Publication
(10) Pub. No.: US 2007/0042951 A1
(43) Pub. Date: Feb. 22, 2007

(54) Pharmaceutical compositions comprising alpha-2-adrenergics and trefoil factor family peptides

(75) Inventors: Orest Olejnik, Coto de Caza, CA (US); Peter G. Bakhit, Huntington Beach, CA (US); Richard Graham, Irvine, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 10/574,523

(22) PCT Filed: Aug. 26, 2004
(86) PCT No.: PCT/US04/27914

§ 371 Date: Mar. 30, 2006

Related U.S. Application Data
(60) Provisional application No. 60/509,955, filed on Oct. 8, 2003.

(51) Int. Cl. A61K 38/17; A61K 31/55; A61K 31/54; A61K 31/4164; A61K 31/498; A61K 31/137

(52) U.S. Cl. 514/12; 514/217.04; 514/227.5; 514/397; 514/389; 514/249; 514/374; 514/649; 514/634

(57) ABSTRACT

Disclosed herein are dosage forms comprising an alpha-2-adrenergic agonist and a trefoil factor family peptide. Related to these dosage forms are methods of treating glaucoma or reducing intraocular pressure and methods of treating gastrointestinal disorders.

### BACKGROUND OF THE INVENTION

1. Field of the Invention

The present invention relates to pharmaceutical compositions. In particular, the present invention relates to compositions comprising an alpha-2-adrenergic agonist and a trefoil factor family peptide.

2. Description of Related Art

Human adrenergic receptors are integral membrane proteins which have been classified into two broad classes, the alpha and the beta adrenergic receptors. Both types mediate the action of the peripheral sympathetic nervous system upon binding of catecholamines, norepinephrine and epinephrine.

Norepinephrine is produced by adrenergic nerve endings, while epinephrine is produced by the adrenal medulla. The binding affinity of adrenergic receptors for these compounds forms one basis of the classification: alpha receptors tend to bind norepinephrine more strongly than epinephrine and much more strongly than the synthetic compound isoproterenol. The preferred binding affinity of these hormones is reversed for the beta receptors. In many tissues, the functional responses, such as smooth muscle contraction, induced by alpha receptor activation are opposed to responses induced by beta receptor binding.

Subsequently, the functional distinction between alpha and beta receptors was further highlighted and refined by the pharmacological characterization of these receptors from various animal and tissue sources. As a result, alpha and beta adrenergic receptors were further subdivided into α₁, α₂, β₁, and β₂ subtypes.

Functional differences between al and a2 receptors have been recognized, and compounds which exhibit selective binding between these two subtypes have been developed. Thus, in WO 92/0073, the selective ability of the R(+) enantiomer of terazosin to selectively bind to adrenergic receptors of the α₁ subtype was reported. The α₁/α₂ selectivity of this compound was disclosed as being significant because agonist stimulation of the α₂ receptors was said to inhibit secretion of epinephrine and norepinephrine, while antagonism of the α₂ receptor was said to increase secretion of these hormones. Thus, the use of non-selective alpha-adrenergic blockers, such as phenoxybenzamine and phentolamine, was said to be limited by their α₂ adrenergic receptor mediated induction of increased plasma catecholamine concentration and the attendant physiological sequelae (increased heart rate and smooth muscle contraction).

For a general background on the α-adrenergic receptors, the reader's attention is directed to Robert R. Ruffolo, Jr., α-Adrenoreceptors: Molecular Biology, Biochemistry and Pharmacology, (Progress in Basic and Clinical Pharmacology series, Karger, 1991), wherein the basis of α₁/α₂ subclassification, the molecular biology, signal transduction, agonist structure-activity relationships, receptor functions, and therapeutic applications for compounds exhibiting α-adrenergic receptor affinity was explored.

The cloning, sequencing and expression of alpha receptor subtypes from animal tissues has led to the subclassification of the α₁ adrenoreceptors into α_{1A}, α_{1B}, and α_{1D}. Similarly, the α₂ adrenoreceptors have also been classified α_{2A}, α_{2B}, and α_{2C} receptors. Each α₂ receptor subtype appears to exhibit its own pharmacological and tissue specificities. Compounds having a degree of specificity for one or more of these subtypes may be more specific therapeutic agents for a given indication than an α₂ receptor panagonist (such as the drug clonidine) or a panantagonist.

British Patent 1 499 485, published Feb. 1, 1978 describes certain thiocarbamide derivatives; some of these are said to be useful in the treatment of conditions such as hypertension, depression or pain.

Some alpha-2-adrenergic compounds, such as brimonidine, are useful for the treatment of glaucoma or the reduction of intraocular pressure. Glaucoma is a disease of the eye characterized by increased intraocular pressure. On the basis of its etiology, glaucoma has been classified as primary or secondary. For example, primary glaucoma in adults (congenital glaucoma) may be either open-angle or acute or chronic angle-closure. Secondary glaucoma results from pre-existing ocular diseases such as uveitis, intraocular tumor or an enlarged cataract.

The underlying causes of primary glaucoma are not yet known. The increased intraocular tension is due to the obstruction of aqueous humor outflow. In chronic open-angle glaucoma, the anterior chamber and its anatomic structures appear normal, but drainage of the aqueous humor is impeded. In acute or chronic angle-closure glaucoma, the anterior chamber is shallow, the filtration angle is narrowed, and the iris may obstruct the trabecular meshwork at the entrance of the canal of Schlemm. Dilation of the pupil may push the root of the iris forward against the angle, and may produce pupilary block and thus precipitate an acute attack. Eyes with narrow anterior chamber angles are predisposed to acute angle-closure glaucoma attacks of various degrees of severity.

Secondary glaucoma is caused by any interference with the flow of aqueous humor from the posterior chamber into the anterior chamber and subsequently, into the canal of Schlemm. Inflammatory disease of the anterior segment may prevent aqueous escape by causing complete posterior synechia in iris bombe, and may plug the drainage channel with exudates. Other common causes are intraocular tumors, enlarged cataracts, central retinal vein occlusion, trauma to the eye, operative procedures and intraocular hemorrhage.

Considering all types together, glaucoma occurs in about 2% of all persons over the age of 40 and may be asymptotic for years before progressing to rapid loss of vision. In cases where surgery is not indicated, topical β-adrenoreceptor antagonists have traditionally been the drugs of choice for treating glaucoma.

Trefoil peptides, or trefoil factor family (TFF) peptides are a class of peptides which comprise a common structural motif, known as the trefoil domain, as part of their structure. The trefoil motif comprises about 20 to about 60 amino acid residues (usually about 40) containing six cysteine residues. The six cysteine residues form three disulfide bridges that complete three loops in the peptide chain so that the roughly 40 residues have a clover-like shape, known as the trefoil domain. TFF-peptides can have one or two trefoil domains per molecule, and may comprise additional amino acid residues which are not part of the trefoil domain. To date, three type of TFF-peptides have been isolated from humans-TFF1 (also known as pS2), TFF2 (also known as SP), and TFF3 (also known as ITF). TFF1 and TFF3 peptides each contain one trefoil domain, while TFF2 peptides contain two trefoil domains. TFF1 and TFF2 peptides are both produced by mucus-producing cells of stomach, while TFF3 peptides are produced by goblet cells of small and large intestine.

All three forms of TFF-peptides are known to be produced in epithelial cells around areas of damage to mucus membrane, suggesting that trefoils have a role in healing injury, particularly to epithelial cells. It is believed that TFF-peptides assist healing by both stabilizing mucus membrane at the injury site and by stimulating repair. It has been shown that TFF-peptides noncovalently link mucin, thus influencing the rheology (e.g. increases viscosity) of mucus gels. [Hauser F, Poulsom R, Chinery R, et al, Proc Natl Acad Sci USA, 1993, vol. 90, pp. 6961-6965; and Babyatsky M W, deBeaumont M, Thim L, Podolky D K, Gastroenterology, 1996, vol. 110, pp. 489-497]. TFF-peptides also appear to be responsible for promoting the migration of epithelial cells to the site of injury, thus stimulating repair. [Göke M, et al, Experimental Cell Research, 2001, vol 264, pp. 337-344; and Playford R J, Journal of the Royal College of Physicians of London, vol 31, pp. 37-40]

In making the above statements, the applicants make no admission as to whether any of the references cited herein are prior art.

### SUMMARY OF THE INVENTION

Disclosed herein are dosage forms comprising an alpha-2-adrenergic agonist and a trefoil factor family peptide. Related to these dosage forms are methods of treating glaucoma or reducing intraocular pressure comprising topically administering an alpha-2-adrenergic agonist and a trefoil factor family peptide to an eye of a mammal suffering from glaucoma. Also related to these dosage forms are methods of treating a gastrointestinal disorder comprising administering an alpha-2-adrenergic agonist and a trefoil factor family peptide to a mammal suffering from said disorder.

### DETAILED DESCRIPTION OF THE INVENTION

A dosage form according to the disclosure herein may be in any physical form, including solid, liquid, and any combination thereof. In one embodiment, the dosage form is a solid of any form, including but not limited to, a powder, a tablet, or a capsule. In another embodiment, the dosage form is a liquid, including but not limited to, a solution, a liquid suspension, or an emulsion. Aside from the case of a suspension of a solid in a liquid, other mixed forms are also contemplated herein. These include emulsions, suspensions, or solutions comprised in a solid material such a solid matrix, a capsule, a gel coating, and the like.

Additionally the manner of administration of the dosage forms according to the disclosure herein may vary. While not intending to limit the scope of the invention in any way, dosage forms disclosed herein may be administered topically, including topically to they eyes; intravenously; orally; rectally; or by any other means convenient for administration of the active compounds to the affected area.

In relation to the methods disclosed herein, the alpha-2-adrenergic agonist and said trefoil factor family peptide may be administered in separate compositions or dosage forms. Alternatively, the alpha-2-adrenergic agonist and said trefoil factor family peptide may be administered in a single composition.

Certain embodiments relate to methods of treating gastrointestinal disorders. While all gastrointestinal diseases are relevant to dosage forms disclosed herein, examples of diseases which are treated or prevented by these dosage forms include comprises Crohn's disease, ulcerative colitis, gastritis, irritable bowel disease and chronic visceral pain. Ulcerative colitis is of particular interest in relation to the dosage forms disclosed herein. Also contemplated herein is the treatment or prevention of irritable bowel disease.

As used herein, the term “alpha-2 adrenergic agonist” includes chemical entities, such as compounds, ions, complexes and the like, that produces a net sympatholytic response, resulting in increased accommodation, for example, by binding to presynaptic alpha-2 receptors on sympathetic postganglionic nerve endings or, for example, to postsynaptic alpha-2 receptors on smooth muscle cells. A sympatholytic response is characterized by the inhibition, diminishment, or prevention of the effects of impulses conveyed by the sympathetic nervous system. The alpha-2 adrenergic agonists of the disclosed herein bind to the alpha-2 adrenergic receptors presynaptically, causing negative feedback to decrease the release of neuronal norepinephrine. Additionally, they also work on alpha-2 adrenergic receptors postsynaptically, inhibiting beta-adrenergic receptor-stimulated formation of cyclic AMP, which contributes to the relaxation of the ciliary muscle, in addition to the effects of postsynaptic alpha-2 adrenergic receptors on other intracellular pathways. Activity at either pre- or postsynaptic alpha-2 adrenergic receptors will result in a decreased adrenergic influence. Decreased adrenergic influence results in increased contraction resulting from cholinergic innervations. Alpha-2 adrenergic agonists also include compounds that have neuroprotective activity. For example, 5-bromo-6-(2-imidozolin-2-ylamino) quinoxaline is an alpha-2-adrenergic agonist which has a neuroprotective activity through an unknown mechanism.

Without limiting the invention to the specific groups and compounds listed, the following is a list of representative alpha-2 adrenergic agonists useful in the compositions and methods disclosed herein: imino-imidazolines, including clonidine, apraclonidine; imidazolines, including naphazoline, xymetazoline, tetrahydrozoline, and tramazoline; imidazoles, including detomidine, medetomidine, and dexmedetomidine; azepines, including B-HT 920 (6-allyl-2-amino-5,6,7,8tetrahydro-4H-thiazolo[4,5-d]-azepine and B-HT 933; thiazines, including xylazine; oxazolines, including rilmenidine; guanidines, including guanabenz and guanfacine; catecholamines and the like. These classes of compounds are well known in the art.

Particularly useful alpha-2-adrenergic agonists include quinoxaline components. In one embodiment, the quinoxaline components include quinoxaline, derivatives thereof and mixtures thereof. One particularly useful class of quinoxaline derivatives is those derivatives comprising (2-imidozolin-2-ylamino) quinoxaline. A special subclass of this group includes 5-halide-6-(2-imidozolin-2-ylamino) quinoxaline. The “halide” of the 5-halide-6-(2-imidozolin-2-ylamino) quinoxaline may be a fluorine, a chlorine, an iodine, or a bromine, to form 5-bromo-6-(2-imidozolin-2-ylamino) quinoxaline. One derivative of quinoxaline that is of particular interest herein is 5-bromo-6-(2-imidozolin-2-ylamino) quinoxaline, or Brimonidine, which is often used in the form of the tartrate salt.

Other useful quinoxaline derivatives are well known. For example, useful derivatives of a quinoxaline include the ones disclose by Burke et al U.S. Pat. No. 5,703,077. See also Danielwicz et al U.S. Pat. No. 3,890,319. Each of the disclosures of Burke et al and Danielwicz et al is incorporated in its entirety by reference herein.

One class of alpha-2-adrenergic agonists which is relatively new in the art is referred to as imidazole-2-thiones for the purposes of this disclosure. These compounds, and methods of preparing them, are described in U.S. patent application Ser. No. 10/153,328, incorporated herein by reference. While not intending to limit the scope of the invention in any way, two examples of useful imidazole-2-thiones compounds for the compositions and methods disclosed herein are compounds 1 and 2, shown below.

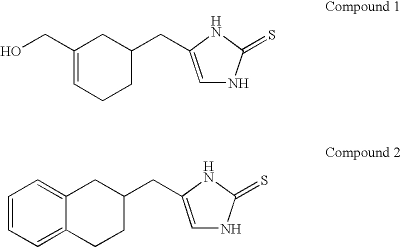

In many cases, the alpha-2-adrenergic agonists, for example the ones listed above, are effective toward activating one or more of alpha-2A-adrenergic receptors, alpha-2B-adrenergic receptors and alpha-2D-adrenergic receptors.

The concentration or amount of the alpha-2-adrenergic agonist used in the dosage forms and methods herein can vary, and is related to the type of dosage form and to the particular use of the alpha-2-adrenergic agonist. Such a determination is well within the ability of one with ordinary skill in the art. While not intending to limit the scope of the invention in any way, in certain embodiments in related to the treatment of glaucoma or the reduction of intraocular pressure, the alpha-2-adrenergic agonist administered at a concentration of from 0.005% to 0.5%. In other embodiments, the alpha-2-adrenergic agonist is administered at a concentration of from 0.02% to 0.2%. In yet other embodiments, the alpha-2-adrenergic agonist is administered at a concentration of about 0.03%. In other embodiments, the alpha-2-adrenergic agonist is administered at a concentration of about 0.1%.

In relation to the treatment of gastrointestinal disorders, the amount or concentration of alpha-2-agonist used can vary. In certain embodiments the alpha-2-adrenergic agonist is administered at a concentration of from 0.1% to 2%. In other embodiments, the concentration of the alpha-2 adrenergic agonist is about 0.6%.

In relation to oral dosage forms, the amount or concentration of alpha-2-agonist used can vary. In certain embodiments the concentration of the alpha-2-adrenergic agonist is from 0.1% to 2%. In other embodiments, the concentration of the alpha-2 adrenergic agonist is about 0.6%.

The term trefoil factor family (TFF) peptide as used herein refers to any peptide, whether natural or synthetic, which comprises the trefoil motif described previously herein. That is, the TFF-peptide comprises a residue comprising from 20 to about 60 amino acids, including six cysteine residues. The cysteine residues form disulfide bonds which cause the peptide residue to have a clover-like shape comprising three loops. The methods of preparing of TFF-peptides, such as recombinant expression of peptides and synthetic peptide synthesis, are well known in the art. For example, methods of preparing TFF-peptides are included in the following references: U.S. Pat. No. 6,525,018; Allen, et. al., J Clin Gastroenterol 1998; 10 (Suppl 1): S93-S98; Ligumsky, et. al., Isr J Med Sci 1986; 22:801-806; Dignass, et. al., J. Clin. Invest., 94, 376-383; Babyatsky, et. al., Gastroenterology, 110, 489-497; Hauser, et. al., Proc. Natl. Acad. Sci. USA, vol. 90, pp. 6961-6965, August 1993; WO 02102403; and WO02085402, incorporated herein by reference. In one embodiment the trefoil factor family peptide is TFF1, TFF2, or TFF3. In another embodiment the trefoil factor family peptide is TFF1 or TFF2. In another embodiment the trefoil factor family peptide is TFF1. In another embodiment the trefoil factor family peptide is TFF2. In another embodiment the trefoil factor family peptide is TFF3.

The amount or concentration of the trefoil factor family peptide used as described herein can vary, and the determination of the proper amount is well within the ability of the ordinary practioner. While not intending to limit the scope of the invention in any way, the particular circumstances in which the trefoil factor family peptide is used may be a relevant consideration in determining the amount or concentration used.

With respect to ophthalmic adminstration, the amount or concentration of the trefoil factor family peptide may vary. In one embodiment, the trefoil factor family peptide administered at a concentration from 0.001% to 1%. In another embodiment, the trefoil factor family peptide administered at a concentration from 0.01% to 0.5%. In another embodiment, the trefoil factor family peptide administered at a concentration from 0.1% to 0.2%. In another embodiment, the trefoil factor family peptide administered at a concentration about 0.15%.

With respect to the treatment of gastrointestinal disorders, the amount or concentration of the trefoil factor family peptide can also vary. In one embodiment, the trefoil factor family peptide is administered at a concentration from 0.1% to 1%. In another embodiment, the trefoil factor family peptide is administered at a concentration of about 0.5%.

With respect to oral dosage forms, the amount or concentration of the trefoil factor family peptide can also vary. In one embodiment, the concentration of the trefoil factor family peptide is from 0.1% to 1%. In another embodiment, the concentration of the trefoil factor family peptide is about 0.5%.

The term “mucoadhesive” used herein means a natural or synthetic component, including macromolecules, polymers, and oligomers, or mixtures thereof, that can adhere to a subject's mucous membrane. Adhesion of mucoadhesives to the mucous membrane occurs primarily through noncovalent interactions, such as hydrogen bonding and Van der Waal forces (Tabor et al., 1977 J. Colloid Interface Sci. 58:2 and Good 1977 J. Colloid Interface Sci. 59:398). Examples of mucoadhesives for use in the embodiments disclosed herein include, but are not limited to, Carbopol®, pectin, alginic acid, alginate, chitosan, hyaluronic acid, polysorbates, such as polysorbate-20, -21, -40, -60, -61, -65, -80, -81, -85; poly(ethyleneglycol), such as PEG-7, -14, -16, -18, -55, -90, -100, -135, -180, 4, -240, -6, -8, -9, -10, -12, -20, or -32; oligosaccharides and polysaccharides, such as Tamarind seed polysaccharide, gellan, carrageenan, xanthan gum, gum Arabic, and dextran; cellulose esters and cellulose ethers; modified cellulose polymers, such as carboxymethylcellulose, hydroxyethylcellulose, hydroxypropyl methylcellulose, hydroxyethyl ethylcellulose; polyether polymers and oligomers, such as polyoxyethylene; condensation products of poly(ethyleneoxide) with various reactive hydrogen containing compounds having long hydrophobic chains (e.g. aliphatic chains of about 12 to 20 carbon atoms), for example, condensation products of poly(ethylene oxide) with fatty acids, fatty alcohols, fatty amides, polyhydric alcohols; polyether compounds, such as poly(methyl vinyl ether), polyoxypropylene of less than 10 repeating units; polyether compounds, such as block copolymers of ethylene oxide and propylene oxide; mixtures of block copolymers of ethylene oxide and propylene oxide with other excipients, for example poly(vinyl alcohol); polyacrylamide; hydrolyzed polyacrylamide; poly(vinyl pyrrolidone); poly(methacrylic acid); poly(acrylic acid) or crosslinked polyacrylic acid, such as Carbomer®, i.e., a homopolymer of acrylic acid crosslinked with either an allyl ether of pentaerythritol, an allyl ether of sucrose, or an allyl ether of propylene. In certain embodiments the mucoadhesive is a polysaccharide.

The term “salt” has the meaning normally understood by those of ordinary skill in the art. A “pharmaceutically acceptable salt” is any salt that retains the activity of the parent compound and does not impart any deleterious or untoward effect on the subject to which it is administered and in the context in which it is administered.

Pharmaceutically acceptable salts of acidic functional groups may be derived from organic or inorganic bases. The salt may be a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

While not intending to limit the scope of the invention in any way, it is often desirable for the pH of such ophthalmic solutions to be maintained between 6.5 and 7.2 with an appropriate buffer system. Various buffers and means for adjusting pH may be used so long as the resulting preparation is ophthalmically acceptable. Accordingly, buffers include acetate buffers, citrate buffers, phosphate buffers and borate buffers. Acids or bases may be used to adjust the pH of these formulations as needed. The formulations may also contain conventional, pharmaceutically acceptable preservatives, stabilizers and surfactants.

Preservatives that may be used in the pharmaceutical compositions disclosed herein include, but are not limited to, benzalkonium chloride, chlorobutanol, thimerosal, phenylmercuric acetate and phenylmercuric nitrate. A useful surfactant is, for example, Polysorbate 80. Likewise, various vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Osmotic agents may be added as needed or convenient. They include, but are not limited to, salts, particularly sodium chloride, potassium chloride, mannitol and glycerin, or any other suitable ophthalmically acceptable tonicity adjustor.

In a similar vein, an ophthalmically acceptable antioxidant for use in the dosage forms described herein, but is not limited to, sodium metabisulfite, sodium thiosulfate, acetylcysteine, butylated hydroxyanisole and butylated hydroxytoluene.

Other excipient components which may be included in the ophthalmic preparations are chelating agents. One useful chelating agent is edetate disodium, although other chelating agents may also be used in place or in conjunction with it.

The non-active ingredients are usually used in the following amounts:

| Ingredient | Amount (% w/v) |
|---|---|
| preservative | 0-0.10 |
| vehicle | 0-40 |
| osmotic agent | 1-10 |
| buffer | 0.01-10 |
| pH adjustor | q.s. pH 4.5-7.5 |
| antioxidant | as needed |
| surfactant | as needed |
| purified water | as needed to make 100% |

The dosage forms disclosed herein are conveniently packaged in forms suitable for metered application, such as in containers equipped with a dropper, to facilitate the application to the eye. Containers suitable for dropwise application are usually made of suitable inert, non-toxic plastic material, and generally contain between about 0.5 and about 15 ml solution.

The foregoing description details specific methods and compositions that can be employed to practice the present embodiments disclosed herein, and represents the best mode contemplated. However, it is apparent for one of ordinary skill in the art that different pharmaceutical compositions may be prepared and used with substantially the same result. Thus, however detailed the foregoing may appear in text, it should not be construed as limiting the overall scope hereof; rather, the ambit of the present invention is to be governed only by the lawful construction of the appended claims.

### EXAMPLE 1

An ophthalmic liquid is formulated according to the composition of Table 1.

TABLE 1

| | | Concentration |
|---|---|---|
| Component | Function | (% wt/wt) |
| Compound 1 | Alpha-2-adrenergic | 0.03 |
| TFF1 | Trefoil factor family peptide | 0.15 |
| Polysorbate 80 | Surfactant | 1 |
| Glycerine | Osmotic agent | 2 |
| Sodium Phosphate | Buffer | 0.1 |
| Water | | QS |

### EXAMPLE 2

An ophthalmic liquid is formulated according to the composition of Table 2.

TABLE 2

| | | Concentration |
|---|---|---|
| Component | Function | (% wt/wt) |
| Compound 2 | Alpha-2-adrenergic | 0.03 |
| TFF1 | Trefoil factor family peptide | 0.15 |
| Polysorbate 80 | Surfactant | 1 |
| Glycerine | Osmotic agent | 2 |
| Sodium Phosphate | Buffer | 0.1 |
| Water | | QS |

### EXAMPLE 3

An ophthalmic liquid is formulated according to the composition of Table 3.

TABLE 3

| | | Concentration |
|---|---|---|
| Component | Function | (% wt/wt) |
| Compound 1 | Alpha-2-adrenergic | 0.03 |
| TFF3 | Trefoil factor family peptide | 0.15 |
| Polysorbate 80 | Surfactant | 1 |
| Glycerine | Osmotic agent | 2 |
| Sodium Phosphate | Buffer | 0.1 |
| Water | | QS |

### EXAMPLE 4

An ophthalmic liquid is formulated according to the composition of Table 4.

TABLE 4

| | | Concentration |
|---|---|---|
| Component | Function | (% wt/wt) |
| Compound 2 | Alpha-2-adrenergic | 0.03 |
| TFF3 | Trefoil factor family peptide | 0.15 |
| Polysorbate 80 | Surfactant | 1 |
| Glycerine | Osmotic agent | 2 |
| Sodium Phosphate | Buffer | 0.1 |
| Water | | QS |

### EXAMPLE 5

An ophthalmic liquid is formulated according to the composition of Table 5.

TABLE 5

| | | Concentration |
|---|---|---|
| Component | Function | (% wt/wt) |
| Compound 1 | Alpha-2-adrenergic | 0.1 |
| TFF3 | Trefoil factor family peptide | 0.15 |
| Polysorbate 80 | Surfactant | 1 |
| Glycerine | Osmotic agent | 2 |
| Sodium Phosphate | Buffer | 0.1 |
| Water | | QS |

### EXAMPLE 6

An ophthalmic liquid is formulated according to the composition of Table 6.

TABLE 6

| | | Concentration |
|---|---|---|
| Component | Function | (% wt/wt) |
| Compound 2 | Alpha-2-adrenergic | 0.1 |
| TFF3 | Trefoil factor family peptide | 0.15 |
| Polysorbate 80 | Surfactant | 1 |
| Glycerine | Osmotic agent | 2 |
| Sodium Phosphate | Buffer | 0.1 |
| Water | | QS |

### EXAMPLE 7

A dosage form of one of the previous examples is administered to a person suffering from glaucoma. Within a short period of time, reduced intraocular pressure, and adverse events are reduced relative to a similar treatment lacking the trefoil factor family peptide.

### EXAMPLE 8

The aqueous liquid suspension formulation of Table 8 is prepared according to the following procedure. All of the ingredients except the trefoil factor family peptide are combined to form a suspension using a milling technique. The trefoil is not milled, but is aseptically added after the milling process is complete.

TABLE 8

| | | Concentration (% |
|---|---|---|
| Component | Function | wt/wt) |
| Compound 1 | Alpha-2-adrenergic | 0.6 |
| TFF3 | Trefoil factor family peptide | 0.5 |
| Hydroxypropylmethyl | Mucoadhesive | 0.2 |
| cellulose E4M | | |
| Pluronic F-127 | Surfactant | 1 |
| Citric Acid | Buffer | 0.1 |
| Water | | QS |

### EXAMPLE 9

The aqueous suspension formulation of Table 9 is prepared according to the procedure of Example 8.

TABLE 9

| | | Concentration (% |
|---|---|---|
| Component | Function | wt/wt) |
| Compound 2 | Alpha-2-adrenergic | 0.6 |
| TFF1 | Trefoil factor family peptide | 0.5 |
| Hydroxypropylmethyl | Mucoadhesive | 0.2 |
| cellulose E4M | | |
| Pluronic F-127 | Surfactant | 1 |
| Citric Acid | Buffer | 0.1 |
| Water | | QS |

### EXAMPLE 10

The liquid suspension formulation of Table 10 is prepared according to the procedure of Example 8.

TABLE 9

| | | Concentration (% |
|---|---|---|
| Component | Function | wt/vol) |
| Compound 1 | Alpha-2-adrenergic | 0.6 |
| TFF1 | Trefoil factor family peptide | 0.5 |
| Hydroxypropylmethyl | Mucoadhesive | 0.2 |
| cellulose E4M | | |
| Pluronic F-127 | Surfactant | 1 |
| Citric Acid | Buffer | 0.1 |
| Water | | QS |

### EXAMPLE 11

The liquid suspension formulation of Table 11 is prepared according to the procedure of Example 8.

TABLE 9

| | | Concentration (% |
|---|---|---|
| Component | Function | wt/wt) |
| Compound 2 | Alpha-2-adrenergic | 0.6 |
| TFF2 | Trefoil factor family peptide | 0.5 |
| Hydroxypropylmethyl | Mucoadhesive | 0.2 |
| cellulose E4M | | |
| Pluronic F-127 | Surfactant | 1 |
| Citric Acid | Buffer | 0.1 |
| Water | | QS |

### EXAMPLE 12

A dosage form according to one of Examples 8-11 is administered orally once a day to a patient suffering from irritable bowel disease. Relief of painful symptoms is experienced by the patient.

What is claimed is:

1. A dosage form comprising an alpha-2-adrenergic agonist and a trefoil factor family peptide.

2. The dosage form of claim 1 wherein said dosage form is a solid.

3. The dosage form of claim 1 wherein said dosage form is a liquid.

4. The dosage form of claim 1 wherein said dosage form is a liquid suspension.

5. The dosage form of claim 4 wherein the concentration of the alpha-2 adrenergic agonist is from 0.1% to 2%.

6. The dosage form of claim 4 wherein the concentration of the alpha-2-adrenergic agonist is about 0.6%.

7. The method of claim 4 wherein the concentration of the trefoil factor family peptide is from 0.1% to 1%.

8. The method of claim 4 wherein the concentration of the trefoil factor family peptide is about 0.5%.

9. The dosage form of claim 1 wherein said alpha-2-adrenergic agonist is selected from the group consisting of imidazole -2-thiones, quinoxaline derivatives, imino-imidazolines, imidazolines, imidazoles, azepines, thiazines, oxazolines, guanidines, catecholamines, and mixtures thereof, or is a pharmaceutically acceptable salt thereof.

10. The dosage form of claim 1 wherein said alpha-2-adrenergic agonist comprises brimonidine or a pharmaceutically acceptable salt thereof.

11. The dosage form of claim 1 wherein said alpha-2-adrenergic agonist comprises an imidazole -2-thione or a pharmaceutically acceptable salt thereof.

12. The dosage form of claim 1 comprising 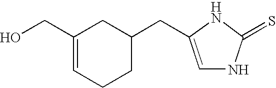
or a pharmaceutically acceptable salt thereof.

13. The dosage form of claim 1 comprising 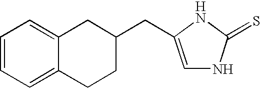
or a pharmaceutically acceptable salt thereof.

14. The dosage form of claim 1 which further comprises a mucoadhesive agent.

15. The dosage form of claim 1 wherein the trefoil factor family peptide is TFF1.

16. The dosage form of claim 1 wherein the trefoil factor family peptide is TFF2.

17. The dosage form of claim 1 wherein the trefoil factor family peptide is TFF3.

18. A method of treating glaucoma or reducing intraocular pressure comprising topically administering an alpha-2-adrenergic agonist and a trefoil factor family peptide to an eye of a mammal suffering from glaucoma.

19. The method of claim 18 wherein said alpha-2-adrenergic agonist and said trefoil factor family peptide are administered in separate compositions.

20. The method of claim 18 wherein said alpha-2-adrenergic agonist and said trefoil factor family peptide are administered in a single composition.

21. The method of claim 18 wherein the alpha-2-adrenergic agonist is administered at a concentration of from 0.005% to 0.5%.

22. The method of claim 18 wherein the alpha-2-adrenergic agonist is administered at a concentration of from 0.02% to 0.2%.

23. The method of claim 22 wherein the alpha-2-adrenergic agonist is administered at a concentration of about 0.03%.

24. The method of claim 22 wherein the alpha-2-adrenergic agonist is administered at a concentration of about 0.1%.

25. The method of claim 18 wherein the trefoil factor family peptide administered at a concentration from 0.001% to 1%.

26. The method of claim 22 wherein the trefoil factor family peptide administered at a concentration from 0.01% to 0.5%.

27. The method of claim 22 wherein the trefoil factor family peptide administered at a concentration from 0.1% to 0.2%.

28. The method of claim 27 wherein the trefoil factor family peptide is administered at a concentration of about 0.15%.

29. The method of claim 18 wherein a mucoadhesive agent is also administered to said patient.

30. The method of claim 18 wherein said trefoil factor family peptide comprises TFF1 or TFF3.

31. A method of treating a gastrointestinal disorder comprising administering an alpha-2-adrenergic agonist and a trefoil factor family peptide to a mammal suffering from said disorder.

32. The method of claim 31 wherein the gastrointestinal disorder comprises Crohn's disease, ulcerative colitis, gastritis, irritable bowel disease and chronic visceral pain.

33. The method of claim 31 wherein the gastrointestinal disorder comprises ulcerative colitis.

34. The method of claim 31 wherein the gastrointestinal disorder comprises irritable bowel disease.

35. The method of claim 31 wherein the trefoil factor family peptide is TFF1.

36. The method of claim 31 wherein the trefoil factor family peptide is TFF3.

37. The method of claim 31 wherein the alpha-2 adrenergic agonist is administered at a concentration from 0.1% to 2%.

38. The method of claim 37 wherein the alpha-2-adrenergic agonist is administered at a concentration of about 0.6%.

39. The method of claim 38 wherein the trefoil factor family peptide is administered at a concentration from 0.1% to 1%

40. The method of claim 39 wherein the trefoil factor family peptide is administered at a concentration of about 0.5%

41. The method of claim 18 wherein said alpha-2-adrenergic agonist and said trefoil factor family peptide are administered in separate dosage forms.

42. The method of claim 18 wherein said alpha-2-adrenergic agonist and said trefoil factor family peptide are administered in a single dosage form.
